(19) United States
(12) Patent Application Publication
(10) Pub. No.: US 2008/0233117 A1
(43) Pub. Date: Sep. 25, 2008

(54) REDUCING TUMOR GROWTH

(75) Inventors: Lewis R. Roberts, Rochester, MN (US); Michael R. Charlton, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/016,062

(22) Filed: Jan. 17, 2008

Related U.S. Application Data
(60) Provisional application No. 60/886,054, filed on Jan. 22, 2007.

(51) Int. Cl. A61K 39/395; A61K 31/70; A61P 43/00

(52) U.S. Cl. 424/133.1; 424/130.1; 424/141.1; 514/44

(57) ABSTRACT

This document provides methods and materials for reducing tumor growth in a mammal. For example, methods and materials for using PLVAP inhibitors to reduce tumor growth in a mammal (e.g., a human) are provided.

### CROSS-REFERENCE TO RELATED APPLICATIONS

This application claims the benefit of U.S. Provisional Application Ser. No. 60/886,054, filed Jan. 22, 2007.

### STATEMENT AS TO FEDERALLY SPONSORED RESEARCH

This invention was made with government support under CA082862 awarded by National Cancer Institute. The government has certain rights in the invention.

### BACKGROUND

1. Technical Field

This document relates to methods and materials involved in reducing tumor growth in a mammal. For example, this document relates to methods and materials that involve using plasmalemma vesicle associated protein (PLVAP) inhibitors to reduce tumor growth in a mammal (e.g., a human).

2. Background Information

Hepatocellular carcinoma (HCC) is the third leading cause of cancer death worldwide, and the incidence of HCC is increasing in the United States. HCCs are typically hypervascular tumors with a rich blood supply that are characterized by neoangiogenesis and vascular invasion. Effective chemotherapy regimens for treating HCC are limited.

### SUMMARY

This document provides methods and materials for reducing tumor growth in a mammal. For example, this document provides methods and materials for using PLVAP inhibitors to reduce tumor growth in a mammal (e.g., a human). The methods and materials provided herein can allow clinicians to reduce tumor growth in patients diagnosed as having one or more tumors. Reducing tumor growth can help cancer patients live longer.

In general, one aspect of this document features a method for reducing tumor growth in a mammal. The method comprises, or consists essentially of, administering a PLVAP inhibitor to the mammal under conditions wherein tumor growth in the mammal is reduced. The mammal can comprise a liver, brain, pancreas, colon, stomach, lung, kidney, ovary, lymph node, skin, breast, or prostate tumor. The mammal can comprise a liver tumor. The mammal can comprise a hepatocellular carcinoma. The PLVAP inhibitor can be an anti-PLVAP antibody. The anti-PLVAP antibody can be a monoclonal antibody. The anti-PLVAP antibody can be an anti-human PLVAP antibody. The anti-PLVAP antibody can be a humanized anti-human PLVAP antibody. The PLVAP inhibitor can comprise nucleic acid that induces RNA interference against nucleic acid encoding a PLVAP polypeptide in the mammal. The PLVAP inhibitor can comprise nucleic acid having a sequence present in SEQ ID NO:2, wherein the sequence is between 15 and 250 nucleotides in length. The sequence can be between 20 and 100 nucleotides in length. The sequence can be between 20 and 50 nucleotides in length. The method can comprise identifying the mammal as having a tumor before administering the PLVAP inhibitor to the mammal. The mammal can be identified as having the tumor using a diagnostic imaging technique. The method can comprise measuring a tumor growth reduction after administering the PLVAP inhibitor to the mammal. The tumor growth reduction can be measured using a diagnostic imaging technique.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting PLVAP signal levels in benign liver tissue and HCC (tumor) tissue, as measured using Affymetrix microarray technology.

FIG. 2 is a graph plotting relative PLVAP expression levels in benign liver tissue and HCC (tumor) tissue, as measured using real-time RT-PCR.

FIG. 3 is a graph plotting relative PLVAP expression levels in benign liver tissue and HCC (tumor) tissue from cirrhotic and non-cirrhotic livers, as measured using real-time PCR.

FIG. 4, left panel is a graph plotting relative PLVAP expression levels in the indicated HCC cell lines, as measured using real-time PCR. FIG. 4, right panel is a Western blot analyzing PLVAP polypeptide expression in the indicated HCC cell lines.

FIG. 5 is a Western blot analyzing PLVAP polypeptide expression in the indicated tissue types.

FIG. 6 is a Western blot analyzing PLVAP polypeptide expression in total cell lysates, and cytosol and membrane fractions, from normal liver (Nml liver), HCC, and benign (Ben) liver tissues.

FIG. 7, top panel is a photomicrograph of a section of normal liver tissue stained with a PLVAP antibody. FIG. 7, bottom panel is a photomicrograph of a tissue section containing HCC tumor tissue located below benign tissue that was stained with a PLVAP antibody.

FIG. 8, top and bottom panels are photomicrographs of sections of benign liver tissue and HCC tumor tissue, respectively, from a tissue microarray stained with a PLVAP antibody.

FIG. 9 is a graph plotting tumor volume of HCC xenographs versus number of days that mice were injected with the mouse anti-PLVAP monoclonal antibody, MECA-32, or with PBS.

FIG. 10 is a graph plotting percentage of mice with tumor volume less than 2000 mm³ versus number of days that the mice were injected with PLVAP monoclonal antibodies or PBS.

FIG. 11 is a listing of a nucleic acid sequence (SEQ ID NO:2) that encodes a human PLVAP polypeptide.

### DETAILED DESCRIPTION

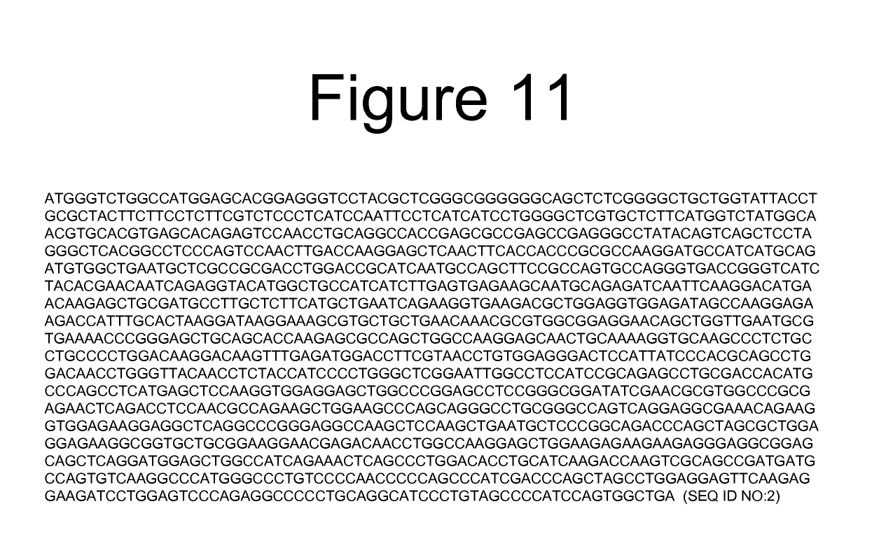

This document provides methods and materials for reducing tumor growth in a mammal. For example, this document provides methods and materials for using PLVAP inhibitors to reduce tumor growth in a mammal (e.g., a human). A PLVAP polypeptide, also referred to as a PV-1 polypeptide or a fenestrated-endothelial linked structure (FELS) polypeptide, can be from any species including, without limitation, dogs, cats, horses, bovine, sheep, monkeys, and humans. Amino acid sequences for PLVAP polypeptides can be as set forth in GenBank gi number 13775238 (see, also, accession number NP_{—}112600) for a human polypeptide, GenBank gi number 14161698 (see, also, accession number NP_{—}115774) for a mouse polypeptide, GenBank gi number 73986220 (see, also, accession number XP_{—}541953) for a dog polypeptide, and GenBank gi number 78369387 (see, also, accession number NP_{—}001030430) for a bovine polypeptide. Nucleic acid sequences that encode a PLVAP polypeptide can be as set forth in GenBank gi number 13775237 (see, also, accession number NM_{—}031310 and FIG. 11) for a human sequence, GenBank gi number 14161697 (see, also, accession number NM_{—}032398) for a mouse sequence, GenBank gi number 73986219 (see, also, accession number XM_{—}541953) for a dog sequence, and GenBank gi number 78369387 (see, also, accession number NM_{—}001035353) for a bovine sequence.

The term “PLVAP inhibitor” as used herein refers to any agent having the ability to inhibit an activity of a PLVAP polypeptide. Examples of activities of a PLVAP polypeptide include participation in the structural assembly of transcellular capillary endothelial pores or fenestrae, which can mediate the exchange of molecules in both directions between blood and tissues, modulation of nuclear shape, and the ability to support tumor growth within a mammal.

As described herein, this document provides methods and materials for reducing tumor growth in a mammal using a PLVAP inhibitor. While not being limited to any particular mode of action, a PLVAP inhibitor (e.g., an anti-PLVAP antibody), once administered to a mammal having a tumor, can interfere with the mammal's vasculature that supplies the tumor. Such interference can result in reduced tumor growth within the mammal. Any type of tumor can be treated using the methods and materials provided herein. For example, liver, brain, pancreas, colon, stomach, lung, kidney, ovary, lymph node, skin, breast, or prostate tumors can be treated with a PLVAP inhibitor such that tumor growth is reduced. In addition, any mammal having such a tumor can be treated including, without limitation, humans, rodents (e.g., rats and mice), goats, pigs, horses, sheep, dogs, cats, cows, and monkeys.

Any appropriate method can be used to determine whether or not a particular agent inhibits the ability of a PLVAP polypeptide to support tumor growth. For example, in vivo tumor growth assays designed to confirm an agent's ability to reduce tumor growth via inhibition of a PLVAP polypeptide activity can be used. Such assays can include the use of anti-PLVAP polypeptide antibodies, siRNA molecules designed to inhibit PLVAP polypeptide expression, and expression cassettes (e.g., regulated expression cassettes) designed to express PLVAP polypeptides. For solid tumors in vivo, the diameter of the tumor can be measured before and after administration and compared to measurements made in controls (e.g., an animal not treated with the agent). Such measurements can be made using a caliper when the tumor has a dermal location. When the tumor occurs in a visceral cavity (e.g., liver or lung) or intracranially (e.g., within the brain), imaging techniques such as contrast enhanced computed tomography (CT) or magnetic resonance imaging (MRI) can be used to measure the size of tumors.

The methods provided herein can include administering a PLVAP inhibitor to a mammal under conditions wherein the PLVAP inhibitor results in a reduced PLVAP polypeptide activity. A reduction in a PLVAP polypeptide activity can result in reduced tumor growth. For example, a PLVAP inhibitor can be used to reduce the growth rate of a tumor by, for example, at least 10, 20, 30, 40, 50, 60, 70, 80, 90, or more percent (e.g., between 10 and 90 percent; between 10 and 75 percent; between 10 and 50 percent; between 50 and 95 percent; between 60 and 95 percent; or between 75 and 95 percent). Any appropriate method can be used to determine the percent reduction in tumor growth within a mammal. For example, imaging techniques such as contrast enhanced CT or MRI can be used to assess tumor growth rates before and after treatment. In some cases, reductions in tumor growth rates can be assessed using histological, biochemical, immunological, or clinical techniques. For example, histological techniques can be used to determine whether or not a tumor expanded into a particular tissue.

Any PLVAP inhibitor can be used to reduce tumor growth in a mammal. For example, anti-PLVAP antibodies can be used to reduce tumor growth in a mammal. In some cases, antisense oligonucleotides, siRNA molecules, RNAi constructs, and PNA oligomers can be designed and used to reduce the level of PLVAP polypeptides expressed. In addition, agents (e.g., small molecule inhibitors) that bind to a PLVAP polypeptide and inhibit a PLVAP polypeptide activity can be used to reduce tumor growth in a mammal. Such agents can be identified using any appropriate method. For example, an organic small molecule capable of inhibiting a PLVAP polypeptide activity can be identified by screening a small molecule library for molecules having the ability to bind to a PLVAP polypeptide and the ability to reduce tumor growth in a manner dependent on PLVAP polypeptide expression.

As described herein, a PLVAP inhibitor can be an anti-PLVAP antibody. For example, in one embodiment, this document provides methods for reducing tumor growth in a mammal by administering an anti-PLVAP antibody to the mammal.

The term “antibody” as used herein refers to intact antibodies as well as antibody fragments that retain some ability to bind an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics.

The antibodies provided herein can be any monoclonal or polyclonal antibody having binding affinity for a PLVAP polypeptide (e.g., a human PLVAP polypeptide). In some cases, an anti-PLVAP antibody can exhibit little, or no, detectable cross reactivity with polypeptides sharing no homology with a PLVAP polypeptide. In some cases, an anti-PLVAP antibody can have the ability to bind a human PLVAP polypeptide in a Western immunoblotting assay when used at a dilution of 1:51,200 or greater.

The antibodies provided herein can be used in immunoassays in liquid phase or bound to a solid phase. For example, the antibodies provided herein can be used in competitive and non-competitive immunoassays in either a direct or indirect format. Examples of such immunoassays include the radioimmunoassay (RIA) and the sandwich (immunometric) assay. As described herein, anti-PLVAP antibodies can be used to reduce tumor growth in a mammal.

Anti-PLVAP antibodies can be obtained from a commercial vender. In some cases, an anti-PLVAP antibody provided herein can be prepared using any appropriate method. For example, any substantially pure PLVAP polypeptide, or fragment thereof, can be used as an immunogen to elicit an immune response in an animal such that specific antibodies are produced. Thus, a human PLVAP polypeptide or a fragment thereof can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art. See, e.g., Green et al., Production of Polyclonal Antisera, in IMMUNOCHEMICAL PROTOCOLS (Manson, ed.), pages 15 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, Current Protocols in Immunology, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, VOL. 10, pages 79 104 (Humana Press 1992).

In addition, methods of in vitro and in vivo multiplication of monoclonal antibodies are well known to those skilled in the art. Multiplication in vitro can be carried out in suitable culture media such as Dulbecco's Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

In some cases, the antibodies provided herein can be made using non-human primates. General techniques for raising therapeutically useful antibodies in baboons can be found, for example, in Goldenberg et al., International Patent Publication WO 91/11465 (1991) and Losman et al., Int. J. Cancer, 46:310 (1990).

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat'l. Acad. Sci. USA, 86:3833 (1989). Techniques for producing humanized monoclonal antibodies are described, for example, by Jones et al., Nature, 321:522 (1986); Riechmann et al., Nature, 332:323 (1988); Verhoeyen et al., Science, 239:1534 (1988); Carter et al., Proc. Nat'l. Acad. Sci. USA, 89:4285 (1992); Sandhu, Crit. Rev. Biotech., 12:437 (1992); and Singer et al., J. Immunol., 150:2844 (1993).

Antibodies provided herein can be derived from human antibody fragments isolated from a combinatorial immunoglobulin library. See, for example, Barbas et al., METHODS: A COMPANION TO METHODS IN ENZYMOLOGY, VOL. 2, page 119 (1991) and Winter et al., Ann. Rev. Immunol., 12: 433 (1994). Cloning and expression vectors that are useful for producing a human immunoglobulin phage library can be obtained, for example, from STRATAGENE Cloning Systems (La Jolla, Calif.). In addition, antibodies provided herein can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody secreting hybridomas. Methods for obtaining human antibodies from transgenic mice are described by Green et al., Nature Genet., 7:13 (1994); Lonberg et al., Nature, 368:856 (1994); and Taylor et al., Int. Immunol., 6:579 (1994).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See, also, Nisonhoff et al., Arch. Biochem. Biophys., 89:230 (1960); Porter, Biochem. J., 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

Other methods of cleaving antibodies, such as separation of heavy chains to form monovalent light heavy chain fragments, further cleavage of fragments, or other enzymatic, chemical, or genetic techniques may also be used provided the fragments retain some ability to bind (e.g., selectively bind) its epitope.

The antibodies provided herein can be substantially pure. The term “substantially pure” as used herein with reference to an antibody means the antibody is substantially free of other polypeptides, lipids, carbohydrates, and nucleic acid with which it is naturally associated in nature. Thus, a substantially pure antibody is any antibody that is removed from its natural environment and is at least 60 percent pure. A substantially pure antibody can be at least about 65, 70, 75, 80, 85, 90, 95, or 99 percent pure.

As described herein, a PLVAP inhibitor can be nucleic acid that induces RNA interference against the mammal's nucleic acid that encodes a PLVAP polypeptide. For example, in one embodiment, this document provides methods for reducing tumor growth in a mammal by administering, to the mammal, nucleic acid that induces RNA interference against nucleic acid encoding a PLVAP polypeptide in the mammal.

Any appropriate method can be used to deliver nucleic acid such as a PLVAP antisense oligonucleotide or PLVAP RNAi construct to a cell. For example, liposomes or lipids can be loaded or complexed with nucleic acid to form nucleic acid-liposome or nucleic acid-lipid complexes. The liposome can be composed of cationic and neutral lipids commonly used to transfect cells in vitro. Cationic lipids can complex (e.g., charge-associate) with negatively charged nucleic acids to form liposomes. Examples of cationic liposomes include lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

In some embodiments, systemic delivery can be optimized using commercially available cationic lipids such as DDAB or DOTAP, each of which can be mixed with a neutral lipid such as DOPE or cholesterol. In some cases, liposomes such as those described by Templeton et al. (Nature Biotechnology, 15:647-652 (1997)) can be used. In other embodiments, polycations such as polyethyleneimine can be used to achieve delivery in vivo and ex vivo (Boletta et al., J. Am Soc. Nephrol. 7: 1728 (1996)).

The mode of delivery can vary with the targeted cell or tissue. For example, nucleic acids can be delivered to lung and liver via the intravenous injection of liposomes since both lung and liver tissue take up liposomes in vivo. In addition, when treating a localized tumor, catheterization in an artery upstream of the affected organ can be used to deliver liposomes containing nucleic acid. This catheterization can avoid clearance of the liposomes from the blood by the lungs and/or liver.

Liposomes containing nucleic acid can be administered parenterally, intravenously, intramuscularly, intraperitoneally, transdermally, excorporeally, or topically. The dosage can vary depending on the species, age, weight, condition of the subject, and the particular compound delivered.

In some embodiments, viral vectors can be used to deliver nucleic acid to a desired target cell. Standard molecular biology techniques can be used to introduce a nucleic acid provided herein into one of the many different viral vectors previously developed to deliver nucleic acid to particular cells. These resulting viral vectors can be used to deliver nucleic acid to the targeted cells by, for example, infection.

An agent having the ability to reduce a PLVAP polypeptide activity (e.g., an anti-PLVAP antibody, an siRNA molecule, an RNAi nucleic acid construct, or a small molecule PLVAP inhibitor) can be administered in amounts and for periods of time that will vary depending upon the nature of the particular tumor, the cancer severity, and the mammal's overall condition. Agents designed to reduce PLVAP polypeptide expression (e.g., siRNA molecules) can be administered in an amount that effectively reduces production of the targeted PLVAP polypeptide. The ability of an agent to effectively reduce production of a PLVAP polypeptide can be assessed, for example, by measuring mRNA or polypeptide levels in a mammal before and after treatment. Any appropriate method can be used to measure mRNA and polypeptide levels in tissues or biological samples such as Northern blots, RT-PCR, immunostaining, ELISAs, and radioimmunoassays. Agents designed to inhibit a PLVAP polypeptide activity by interacting with a PLVAP polypeptide can be administered in an amount that effectively inhibits a PLVAP polypeptide activity or reduces tumor growth. The ability of an agent to inhibit effectively a PLVAP polypeptide activity can be assessed, for example, by using an activity assay such as measurement of the transepithelial electrical resistance (TER) across a monolayer of vascular endothelial cells. The endothelial cells can be seeded on Falcon 12 well cell culture inserts (BD Biosciences) previously coated with a thin layer of a collagen/Matrigel mixture. The inserts can be monitored each day for confluence and electrical resistance using a Millicel-ERS ohm-voltmeter (Millipore Corporation, Bedford, Mass.). The TER values can be calculated by converting to ohm×cm².

Dosing is generally dependent on the severity and responsiveness of the tumor to be treated, with the course of treatment lasting from several days to several months, or until a cure is effected or a diminution of the tumor's state is achieved. Routine methods can be used to determine optimum dosages, dosing methodologies, and repetition rates. Optimum dosages can vary depending on the relative potency of individual compounds, and can generally be estimated based on EC₅₀ values found to be effective in in vitro and/or in vivo animal models. Typically, dosage is from about 0.01 μg to about 100 g per kg of body weight, and can be given once or more daily, weekly, or even less often. Following successful treatment, it may be desirable to have the mammal undergo maintenance therapy to prevent recurrence.

Any appropriate method can be used to formulate and subsequently administer a composition containing one or more PLVAP inhibitors (e.g., an anti-PLVAP antibody, an siRNA molecule, or a small molecule PLVAP inhibitor). For example, compositions containing one or more PLVAP inhibitors provided herein can be admixed, encapsulated, conjugated, or otherwise associated with other molecules such as, for example, liposomes, receptor targeted molecules, oral formulations, rectal formulations, or topical formulations for assisting in uptake, distribution, and/or absorption.

Compositions containing one or more PLVAP inhibitors provided herein can contain one or more pharmaceutically acceptable carriers. A “pharmaceutically acceptable carrier” is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, without limitation, water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

A composition can be administered by a number of methods depending upon whether local or systemic treatment is desired and upon the area to be treated. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, a composition can be administered by injection or infusion into the cerebrospinal fluid, preferably with one or more agents capable of promoting penetration across the blood-brain barrier.

Compositions for topical administration include, for example, sterile and non-sterile aqueous solutions, non-aqueous solutions in common solvents such as alcohols, or solutions in liquid or solid oil bases. Such solutions also can contain buffers, diluents, and other suitable additives. Compositions for topical administration can be formulated in the form of transdermal patches, ointments, lotions, creams, gels, drops, suppositories, sprays, liquids, and powders. Conventional pharmaceutical carriers, aqueous, powder or oily bases, thickeners, and the like can be added.

Compositions for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Compositions for parenteral, intrathecal, or intraventricular administration can include, for example, sterile aqueous solutions, which also can contain buffers, diluents, and other suitable additives (e.g., penetration enhancers, carrier compounds, and other pharmaceutically acceptable carriers).

The invention will be further described in the following examples, which do not limit the scope of the invention described in the claims.

### EXAMPLES

### Example 1

### Expression Profiling of HCC Tissues

To identify tumor markers, genes and pathways involved in the pathogenesis of hepatocellular carcinoma (HCC), high density oligonucleotide microarray technology was used to analyze seven HCV-induced HCCs, six HBV-induced HCCs, and seven HCCs from patients with no known risk factors for HCC. The Affymetrix Human Genome U95A-E array set (Affymetrix, Santa Clara, Calif.) was used. This set of five arrays allows expression profiling of more than 63,000 genes and expressed sequence tags (ESTs). Paired HCC and benign liver tissues adjacent to the HCCs were collected from patients undergoing resection of HCCs, and snap frozen in liquid nitrogen. Most of the HCC tissues that were profiled had volume fractions of tumor cells ranging from about 70-90%. Total RNA was extracted from the tissue specimens using the Qiagen RNeasy Mini Kit (Valencia, Calif.). Purified RNA was quantitated and assessed for quality by formaldehyde agarose gel electrophoresis and by separation on an Agilent 2100 Bioanalyzer. For each oligonucleotide chip, five μg of total RNA were reverse-transcribed and labeled by in vitro transcription in the presence of biotin to produce biotinylated cRNA. Hybridization was performed at 45° C. for 16 hours in a rotary oven, followed by washing and staining of each array. Each chip was then scanned two times, and the results analyzed using Affymetrix and Spotfire software.

To determine the reproducibility and variation inherent in the technique, a set of two duplicate experiments was performed, using benign and tumor tissue from patients 81 and 89. In the first experiment (patient 81), a total amount of 80 μg of total RNA was transcribed, labeled, and fragmented in one single reaction at one time. Duplicate chips were hybridized with aliquots of the fragmented cRNA, washed, stained and scanned together. In the second experiment (patient 89), two separate aliquots of 40 μg of total RNA were transcribed, labeled and fragmented on different days. The duplicate chips were also hybridized separately and scanned on different days. The results were analyzed using Affymetrix GeneChip® software version 5 to obtain the signal (expression value) and detection call (present, absent or marginal). As expected, the overall variation in signal values and detection calls between the two different samples was greater for the samples transcribed, labeled, hybridized and scanned on different days than for the samples processed on the same day. The variation in detection calls between samples was low (2.6%).

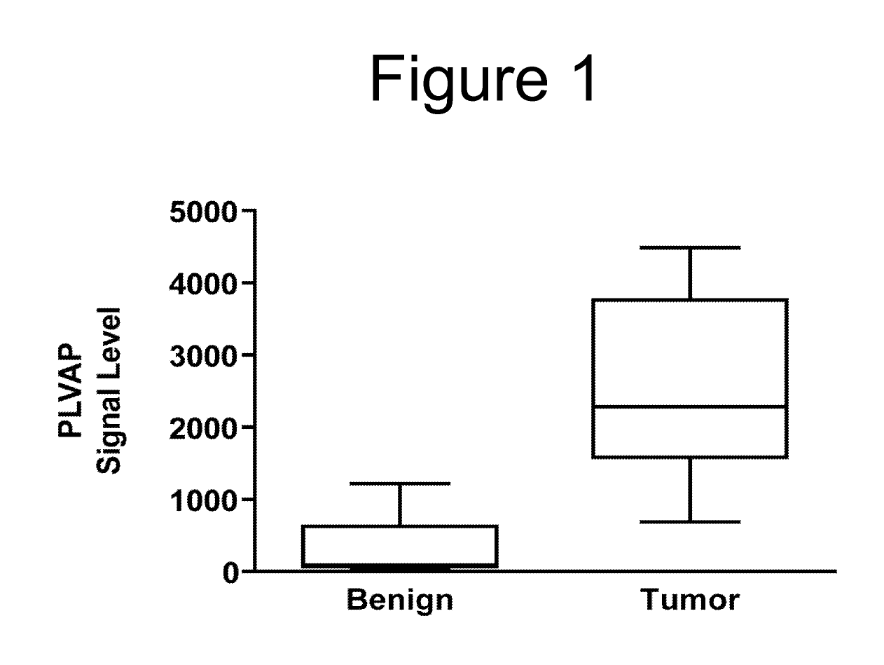

To identify genes and ESTs having increased or decreased expression in HCC, gene expression values for all 63,154 genes and ESTs represented on the microarrays were log-transformed and sorted based on the standard deviation of expression values across all samples. Genes having the 1000 largest standard deviations were ranked in two groups depending on whether they showed increased or decreased expression in tumors as compared to the adjacent benign tissue. Genes that showed the largest differences in the greatest number of HCCs were ranked the highest. One of the top-ranked genes, which showed markedly increased expression in all the HCCs examined, was PLVAP (GenBank EST AI660921), also referred to as PV-1 and fenestrated-endothelial linked structure protein (FELS). This EST was present on the U95D chip. The signal levels of PLVAP from the Affymetrix arrays are presented in FIG. 1 (left panel).

HCC and benign tissues also were analyzed using oligo microarrays produced at the Advanced Technology Center at the National Cancer Institute. HCC and adjacent benign tissues were collected from 139 individuals from two ethnic groups (61 Chinese and 78 white) who were undergoing surgical treatment for HCC. The median duration of follow up was 23.4 months. During this period, 74 individuals died. The median age of the individuals was 57, and 73.3% were male. Of the 78 white individuals, 17 underwent liver transplantation, and 9 received palliative treatment. Data from these 26 individuals were not included in the analysis of survival or tumor recurrence.

The tissue samples were snap frozen in liquid nitrogen. Total RNA was extracted using the Qiagen RNeasy Mini Kit or Trizol (Invitrogen), and the RNA was purified using Qiagen columns. The Human Array-Ready Oligo Set (Version 2.0) containing 70-mer probes of 21,329 genes was obtained from Qiagen, and oligo microarrays were produced at the Advanced Technology Center at the National Cancer Institute. Twenty μg of total RNA were used to generate fluorescently (Cy-5 or Cy-3) labeled cDNA molecules. At least two hybridizations were carried out for each tissue using a dye-swap strategy to eliminate dye-labeling bias as described elsewhere (Stan et al., Mol Biol Cell, 15(8):3615-30 (2004)). Expression ratios of each gene from duplicate experiments were then averaged (Stan et al., Mol Biol Cell, 15(8):3615-30 (2004); Niemela et al., Blood, 106(10):3405-9 (2005); Carson-Walter et al., Clin Cancer Res., 11(21):7643-50 (2005)). These data further confirmed the increase in PLVAP expression in HCCs.

### Example 2

### Validation of PLVAP Expression Using Quantitative Real-Time PCR

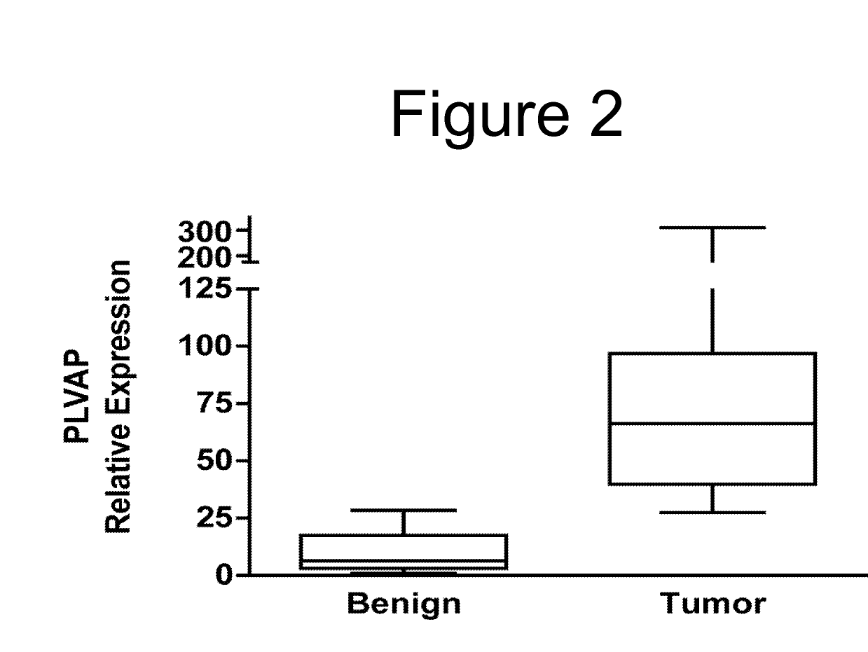

The change in PLVAP gene expression observed in the microarray experiments was validated using quantitative PCR. Primers specific for PLVAP cDNA were designed, and quantitative PCR was performed with cDNA prepared from 19 pairs of HCC and adjacent benign tissues. PLVAP levels were normalized to 18S ribosomal RNA levels in each tissue. Normal kidney tissue, which expresses PLVAP in glomerular capillaries, was used as a positive control. The tumors had significantly increased PLVAP expression compared to the adjacent benign tissue (FIG. 2), confirming the relative difference in gene expression observed in the Affymetrix microarray experiments (FIG. 1, right panel).

In a second validation experiment, PLVAP expression was analyzed in 33 pairs of HCC tumors and adjacent benign tissues using quantitative PCR. Total RNA was extracted from each tissue specimen, and four μg of each sample of total RNA were used to prepare cDNA with the Superscript First Strand Synthesis System (Invitrogen, Carlsbad, Calif.). Quantitative PCR was performed using cDNA from each sample, 2× TaqMan Universal Master Mix (ABI catalog number 4326708) and PLVAP Assay on Demand Gene Expression Kit (ABI), according to the manufacturer's instructions, in 96 well plates with a final reaction volume of 25 μL. Assay on Demand products consist of a 200× mix of unlabeled PCR primers and TaqMan MGB probe FAM dye-labeled. Thermal cycler conditions were as follows: initial set-up at 95° C. for 10 minutes, and then 40 cycles of 95° C. for 15 seconds and 60° C. for one minute. For each set of samples analyzed, a standard curve of known copy number was prepared using plasmid DNA of the extracellular region of PLVAP cloned into the vector pQE81 (Qiagen). As an internal control, identical separate 96 well plates were tested using 18S ribosomal RNA Assay on Demand. Reactions were run on an ABI 7300 Sequence Detection System, and the results were analyzed using ABI 7300 software. Expression of PLVAP was normalized to that of 18s rRNA.

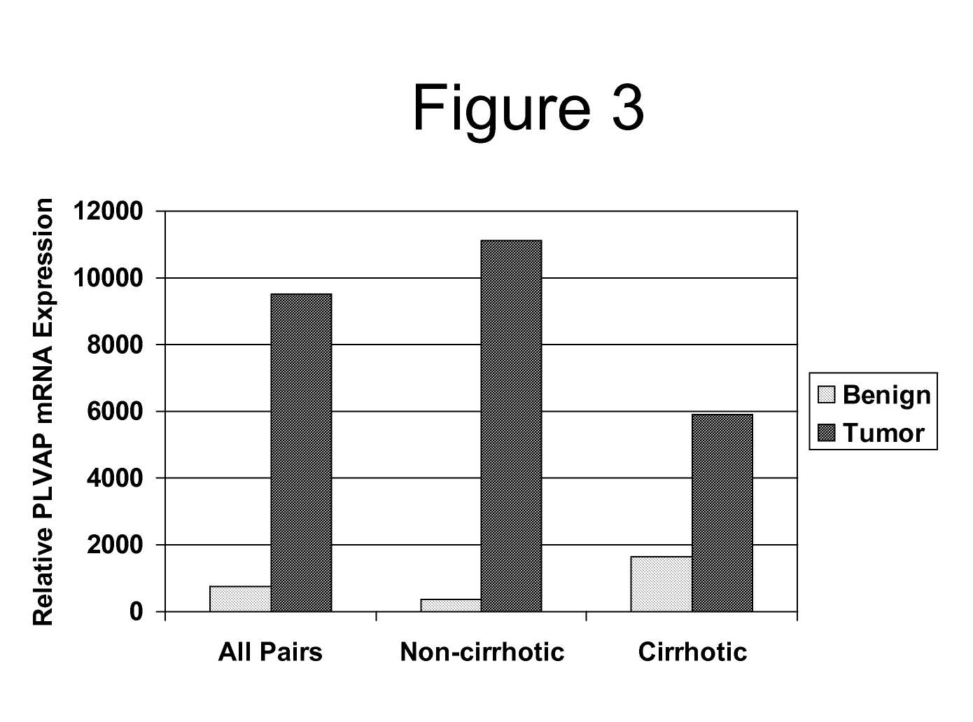

PLVAP expression was significantly increased in HCCs, ranging from a 0.88 fold expression to 284 fold over-expression with the average expression being 28 fold higher in the tumors compared to their adjacent benign tissues (FIG. 3). The ratio of PLVAP over-expression was compared to patient survival, cirrhosis, and hepatitis B and C to determine whether there was a correlation. Expression of PLVAP in benign tissues from patients having cirrhosis was higher than in benign tissues from patients that did not have cirrhosis (FIG. 3). PLVAP expression levels in the tumors from patients not having cirrhosis were higher than PLVAP expression levels in tumors from patients with cirrhosis (FIG. 3).

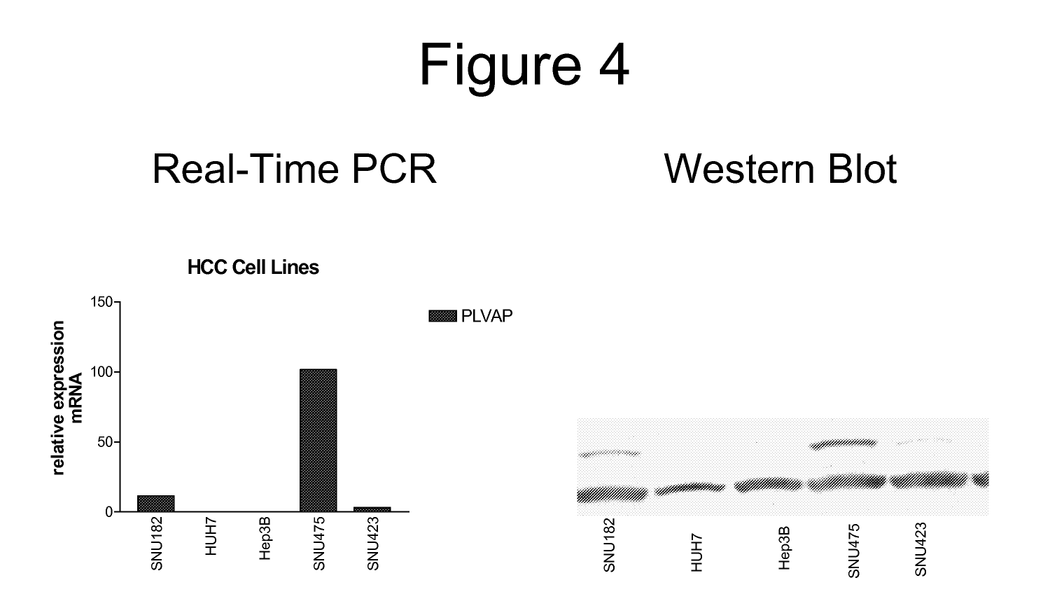

Expression of PLVAP was also examined in several HCC cell lines (SNU182, SNU475, SNU423, HUH7 and Hep3B) using quantitative PCR. The HCC cell lines were obtained from the American Type Culture Collection (ATCC). RNA was extracted from the cell lines using RNeasy Mini Kit (Qiagen). PLVAP expression was observed in the SNU182, SNU475 and SNU423 cell lines, but not in the HUH7 and Hep3B cell lines (FIG. 4, left panel).

### Example 3

### Analysis of PLVAP Polypeptide Expression

The synthetic peptide CPIDPASLEEFKRKILESQRPPAGI (corresponding to amino acid residues 412-435 of the sequence set forth in GenBank accession number NM_{—}031310; SEQ ID NO: 1) was used as an antigen in rabbit to produce polyclonal antibody against human PLVAP (Covance, Princeton, N.J.). The antibody was purified from the rabbit serum using a SulfoLink Column (Pierce, Rockford, Ill.) coupled with the synthetic peptide. The polyclonal antibody directed against human PLVAP was called the PLVAP412 antibody.

Proteins were extracted from HCC cell lines with a lysis buffer containing 25 mM Hepes pH 7.2, 250 mM sucrose, 2 mM MgCl, 0.1% NP40, 1 mM phenylmethylsulfonyl fluoride and a cocktail of protease inhibitors (Calbiochem). The cells were incubated on ice for 30 minutes with vortexing every 10 minutes, and were then centrifuged at 15,000×g for 10 minutes at 4° C. The supernatant was collected and the concentration of protein was determined using a Bio-Rad DC Protein Assay (Catalog number 500-0116).

Benign liver tissue and tumor tissue pairs from four patients diagnosed with hepatocellular carcinoma were obtained after surgical resection. Protein was extracted for immunoblotting. The tissues were homogenized on ice in a lysis buffer containing 25 mM Hepes pH 7.2, 250 mM sucrose, 2 mM MgCl, 0.1% NP40, 1 mM phenylmethylsulfonyl fluoride and a cocktail of protease inhibitors (Calbiochem). The homogenized tissues were centrifuged at 500×g for 30 minutes at 4° C. The supernatant, which was referred to as the total protein lysate, was retained, and the protein concentration was determined using a Bio-Rad DC Protein Assay.

To separate membrane and cytosolic fractions from the total protein lysate, the supernatant was further centrifuged at 100,000×g for 90 minutes at 4° C. and divided into membrane and cytosolic fractions. The membranes were solubilized in 50 mM Tris-HCl (pH 6.8) with 0.5% SDS. The protein concentration of these fractions was determined using a Bio-Rad DC Protein Assay (Hnasko et al., Journal of Endocrinology, 175:649-661 (2002)).

Twenty μg of protein from each of the cell lines and the four tissue pairs were separated under denaturing conditions on a 10% polyacrylamide gel and transferred to a PVDF membrane. The membrane was blocked in 10% non-fat milk in PBS and probed with the PLVAP412 polyclonal antibody diluted in PBS with 3% Tween (1:500 dilution for the cell lines and 1:3000 for the tissues). Goat anti-rabbit IgG (Gamma) HRP (Biosource) diluted in PBS with 3% Tween (1:5000 dilution for cell lines and 1:8000 dilution for tissues) was used as the secondary antibody. Immunoreactive bands were visualized on X-ray film using Amersham's ECL detection reagents.

PLVAP appeared as a band at 50 to 60 kDa on a reducing gel. Occasionally, bands appeared at about 120 kDa, which were dimers of PLVAP, and at about 35 kDa, which were most likely degraded polypeptide products.

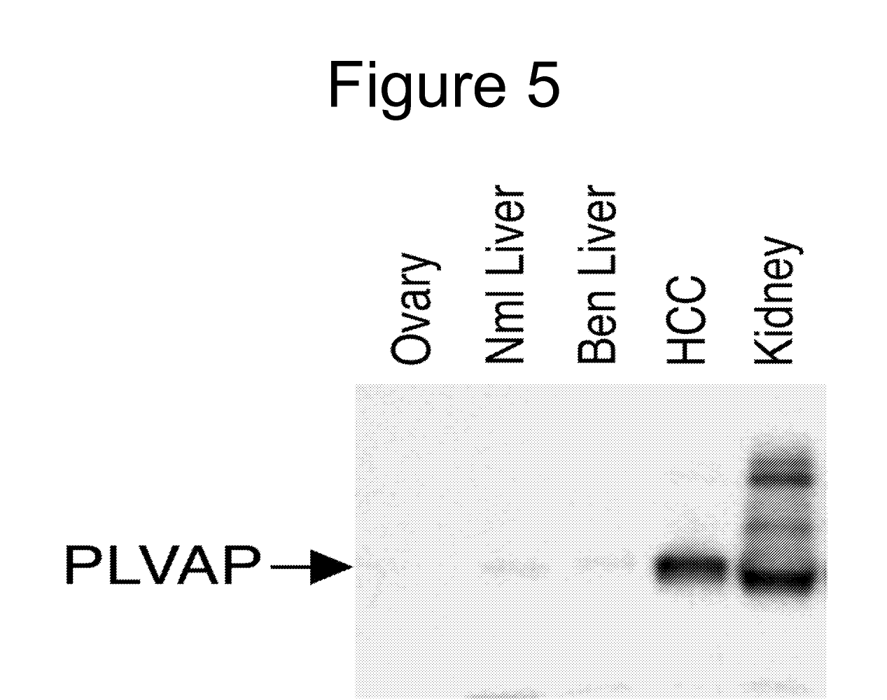

Total protein lysates from normal liver, four HCC tumors, and their four adjacent benign tissues were analyzed by immunoblotting as described above. PLVAP polypeptide was highly over-expressed in all of the HCC tumors as compared to their adjacent benign tissues. Very little PLVAP polypeptide was observed in normal liver (FIG. 5). Of the four pairs of HCC and benign tissues, one patient had no cirrhosis, and three patients had cirrhosis.

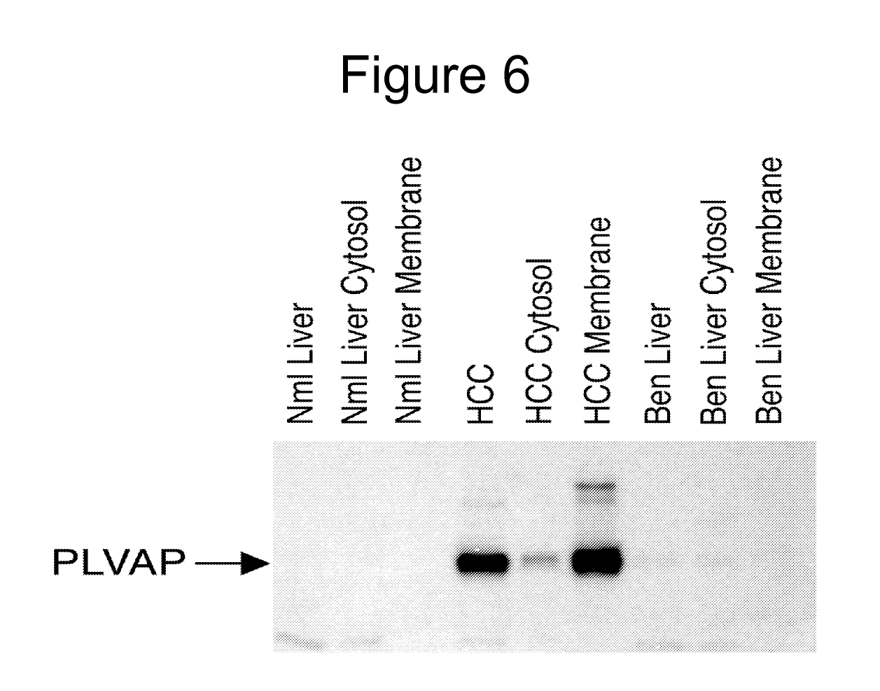

Different protein fractions (total lysate, cytosolic, and membrane) of normal liver, an HCC tumor, and the adjacent benign liver tissue also were analyzed by immunoblotting as described above. PLVAP polypeptide was highly expressed in the HCC tumor, with most of the protein being found in the membrane fraction. A very low level of PLVAP polypeptide expression was observed in the normal liver and in the benign tissue (FIG. 6).

Protein extracts from HCC cell lines also were analyzed by immunoblotting with the PLVAP polyclonal antibody as described above. Expression of PLVAP polypeptide was observed in extracts from SNU182, SNU475 and SNU423 cells (FIG. 4, right panel). However, expression of PLVAP polypeptide was not observed in extracts from HUH 7 and Hep3B cells (FIG. 4, right panel). These cell lines are derived from the malignant epithelial hepatocellular component of HCCs with no endothelium component. The results of these experiments suggested, therefore, that PLVAP may be expressed in the endothelial cell component of HCCs.

### Example 4

### Immunohistochemical Analysis of PLVAP Polypeptide Expression

Paraffin-embedded tissue sections were prepared using the four pairs of matched HCC and benign tissues used in immunoblotting, as described in Example 3. The tissue sections were stained with the PLVAP412 polyclonal antibody at a dilution of 1:500 (and 1:1000) using the EDTA/Dual+/DAB+ retrieval method.

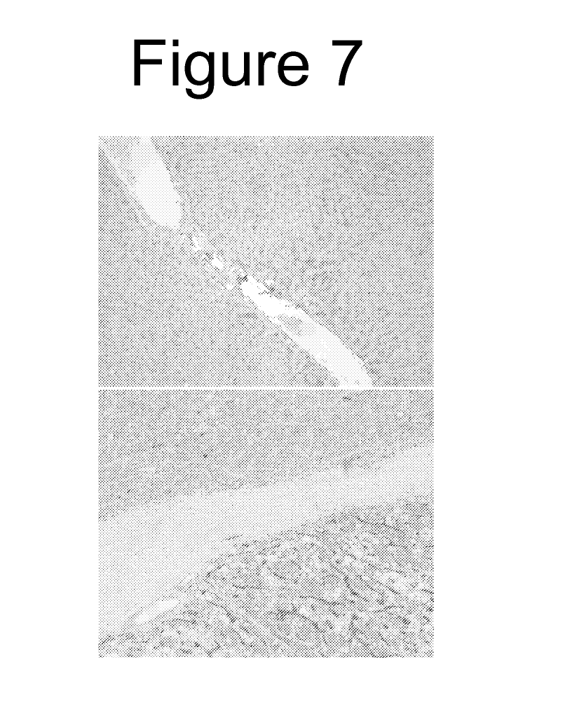

Staining was observed in the vascular regions (e.g., tumor microvessels) of the HCC tumor tissues analyzed by immunohistochemistry with the PLVAP412 polyclonal antibody (FIG. 7). Little staining was observed in the benign tissue, and no staining was observed in normal liver (FIG. 7).

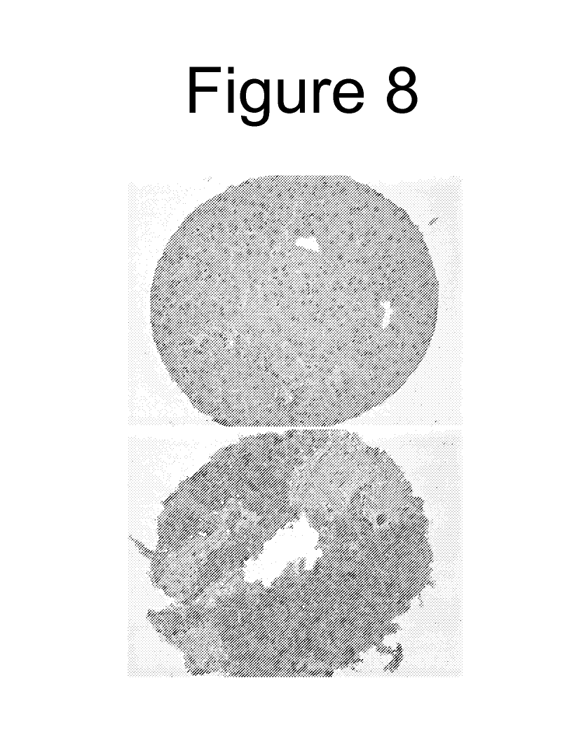

Tissue microarrays containing three 1 mm cores from HCC and three 1 mm cores of adjacent benign liver tissue from each of 210 patients also were stained using the PLVAP412 polyclonal antibody. PLVAP staining was observed only in the vascular regions of the tumor (FIG. 8). These results indicated that PLVAP is a marker for the microvascular endothelium of HCC tumors.

### Example 5

### In Vivo MECA-32 Antibody Trial

A hybridoma cell line producing the mouse anti-PLVAP monoclonal antibody, MECA-32, was obtained from the Developmental Studies Hybridoma Bank, developed under the auspices of the NICHD and maintained by the University of Iowa, Department of Biological Sciences, Iowa City, Iowa. MECA-32 antibody was prepared from the hybridoma cells by the Antibody Core Facility at Mayo Clinic, Rochester, Minn.

Twenty nude mice were randomly assigned to two groups of ten. Each mouse was injected subcutaneously in the right flank with five million HUH7 cells. Tumors were allowed to become established and grow until the tumor volumes were between 150-300 mm³. Tumor volume was calculated according to the formula: TV=[(A×B̂2)/2]. When the tumors were between 150-300 mm³, ten animals in one group were injected intraperitoneally with 0.2 mg/day of the mouse anti-PLVAP monoclonal antibody, MECA-32. As controls, the ten animals in the other group were treated with PBS. The animals were injected every day for 14 days, and tumor volume was also measured every day. At the end of the 14 day antibody/control treatment period, at least five mice from each group were sacrificed, including all mice with tumors having a volume equal to or greater than 4000 mm³. The rest of the mice were chosen at random. The remaining mice were followed for another two weeks, and tumor volume was measured daily. If the tumor size of a mouse reached 4000 mm³ before the end of this two week period, the mouse was sacrificed. At the end of the two weeks, all remaining mice were sacrificed. When an animal was sacrificed, the tumors were saved for examination by H&E staining and immunohistochemistry.

Proteins were also isolated from the tumors to evaluate the presence of cell growth and tumor angiogenesis-related proteins. Proteins from the xenografts were isolated with the same lysis buffer and procedure used for the human tissue samples, as described in Example 3. Twenty μg of protein were analyzed by Western blotting. Separated polypeptides were transferred to a PVDF membrane, which was blocked with 10% milk in PBS and probed with the MECA-32 antibody, at a dilution of 1:1000, and a secondary goat anti-rat IgG HRP antibody (Sigma A9037), at a dilution of 1:2000 in PBS with 0.2% Tween. Western blots were also probed with total ERK1/2 (Biosource International, Camarillo, Calif.), phospho-ERK (Biosource), PDGF-R beta, phosphor PDGF-R beta, and VEGF-R2 antibodies.

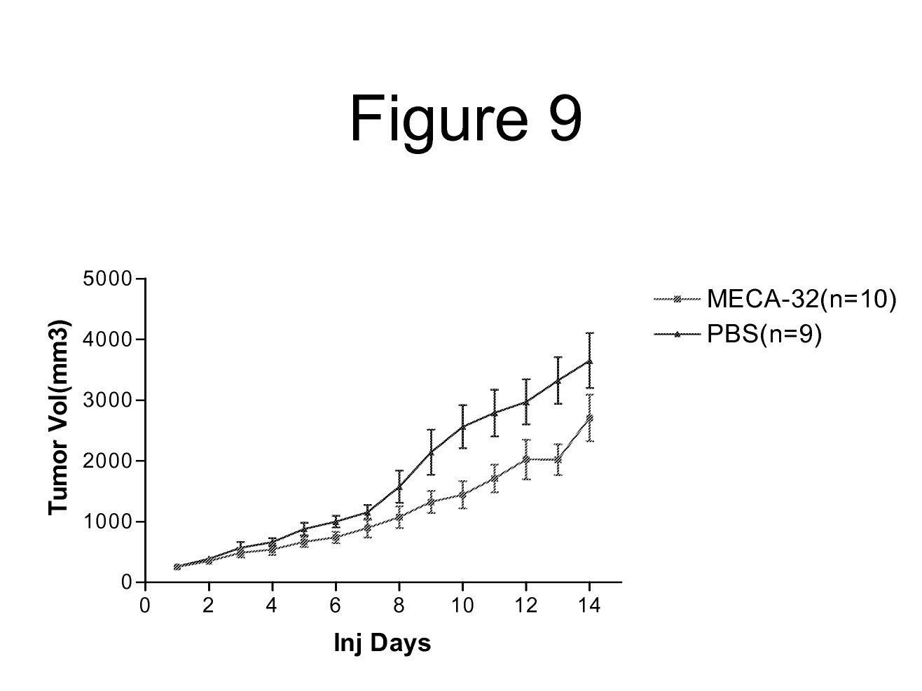

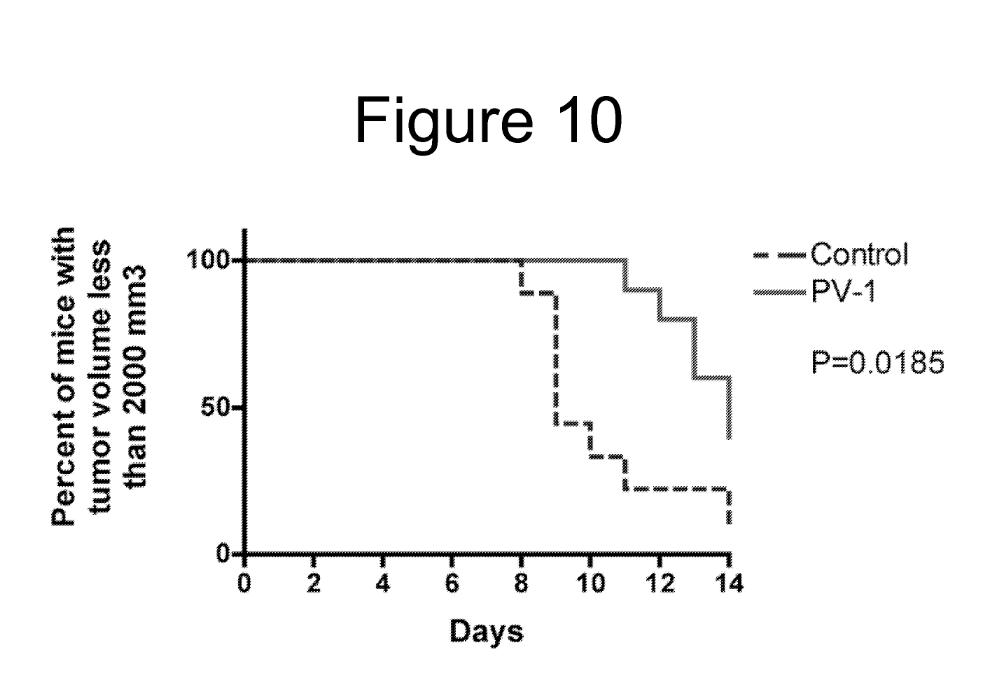

The growth of Huh7 xenographs, produced subcutaneously in nude mice, was delayed significantly after two weeks of intraperitoneal administration of a monoclonal anti-PLVAP antibody targeting the mouse PLVAP polypeptide, when compared to animals bearing similar sized xenografts treated with PBS diluent (FIGS. 9 and 10).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing tumor growth in a mammal, wherein said method comprises administering a PLVAP inhibitor to said mammal under conditions wherein tumor growth in said mammal is reduced.

2. The method of claim 1, wherein said mammal comprises a liver, brain, pancreas, colon, stomach, lung, kidney, ovary, lymph node, skin, breast, or prostate tumor.

3. The method of claim 1, wherein said mammal comprises a liver tumor.

4. The method of claim 1, wherein said mammal comprises a hepatocellular carcinoma.

5. The method of claim 1, wherein said PLVAP inhibitor is an anti-PLVAP antibody.

6. The method of claim 5, wherein said anti-PLVAP antibody is a monoclonal antibody.

7. The method of claim 5, wherein said anti-PLVAP antibody is an anti-human PLVAP antibody.

8. The method of claim 5, wherein said anti-PLVAP antibody is a humanized anti-human PLVAP antibody.

9. The method of claim 1, wherein said PLVAP inhibitor comprises nucleic acid that induces RNA interference against nucleic acid encoding a PLVAP polypeptide in said mammal.

10. The method of claim 9, wherein said PLVAP inhibitor comprises nucleic acid having a sequence present in SEQ ID NO:2, wherein said sequence is between 15 and 250 nucleotides in length.

11. The method of claim 10, wherein said sequence is between 20 and 100 nucleotides in length.

12. The method of claim 10, wherein said sequence is between 20 and 50 nucleotides in length.

13. The method of claim 1, wherein said method comprises identifying said mammal as having a tumor before administering said PLVAP inhibitor to said mammal.

14. The method of claim 13, wherein said mammal is identified as having said tumor using a diagnostic imaging technique.

15. The method of claim 1, wherein said method comprises measuring a tumor growth reduction after administering said PLVAP inhibitor to said mammal.

16. The method of claim 15, wherein said tumor growth reduction is measured using a diagnostic imaging technique.


SEQUENCE LISTING (2 sequences)

SEQ ID NO: 1 - protein, 25 aa, Artificial Sequence
  Synthetically generated peptide
CPIDPASLEEFKRKILESQRPPAGI

SEQ ID NO: 2 - DNA, 1329 bp, Homo sapiens
atgggtctggccatggagcacggagggtcctacgctcgggcggggggcagctctcggggctgctggtattacctgcgctacttcttcctcttcgtctccctcatccaattcctcatcatcctggggctcgtgctcttcatggtctatggcaacgtgcacgtgagcacagagtccaacctgcaggccaccgagcgccgagccgagggcctatacagtcagctcctagggctcacggcctcccagtccaacttgaccaaggagctcaacttcaccacccgcgccaaggatgccatcatgcagatgtggctgaatgctcgccgcgacctggaccgcatcaatgccagcttccgccagtgccagggtgaccgggtcatctacacgaacaatcagaggtacatggctgccatcatcttgagtgagaagcaatgcagagatcaattcaaggacatgaacaagagctgcgatgccttgctcttcatgctgaatcagaaggtgaagacgctggaggtggagatagccaaggagaagaccatttgcactaaggataaggaaagcgtgctgctgaacaaacgcgtggcggaggaacagctggttgaatgcgtgaaaacccgggagctgcagcaccaagagcgccagctggccaaggagcaactgcaaaaggtgcaagccctctgcctgcccctggacaaggacaagtttgagatggaccttcgtaacctgtggagggactccattatcccacgcagcctggacaacctgggttacaacctctaccatcccctgggctcggaattggcctccatccgcagagcctgcgaccacatgcccagcctcatgagctccaaggtggaggagctggcccggagcctccgggcggatatcgaacgcgtggcccgcgagaactcagacctccaacgccagaagctggaagcccagcagggcctgcgggccagtcaggaggcgaaacagaaggtggagaaggaggctcaggcccgggaggccaagctccaagctgaatgctcccggcagacccagctagcgctggaggagaaggcggtgctgcggaaggaacgagacaacctggccaaggagctggaagagaagaagagggaggcggagcagctcaggatggagctggccatcagaaactcagccctggacacctgcatcaagaccaagtcgcagccgatgatgccagtgtcaaggcccatgggccctgtccccaacccccagcccatcgacccagctagcctggaggagttcaagaggaagatcctggagtcccagaggccccctgcaggcatccctgtagccccatccagtggctga